SEQUENCE LISTING (28 sequences)

SEQ ID NO: 1 - protein, 1153 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 2 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 3 - protein, 5 aa, Synthetic construct
NYLIE

SEQ ID NO: 4 - protein, 17 aa, Synthetic construct
VINPGSGGTNYNEKFKG

SEQ ID NO: 5 - protein, 11 aa, Synthetic construct
GDDYDDYTMDY

SEQ ID NO: 6 - protein, 11 aa, synthetic construct
RASQSISNNLH

SEQ ID NO: 7 - protein, 7 aa, synthetic construct
YASQSMS

SEQ ID NO: 8 - protein, 9 aa, synthetic construct
QQSNSWPLT

SEQ ID NO: 9 - protein, 447 aa, synthetic construct
QVQLQQSGAELVRPGTSVKVSCKASGYAFINYLIEWVKQRPGQGLEWIGVINPGSGGTNYNEKFKGKATLTADKSSSTAYMQLSSLTSDDSAVYFCARGDDYDDYTMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIVLSQSPATLSVTPGDSVSLSCRASQSISNNLHWYQQKSHESPRLLIKYASQSMSGIPSRFSGSGSGTDFTLSINSVETEDFGMYFCQQSNSWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 447 aa, synthetic construct
QVQLQQSGAELKRPGSSVKVSCKASGYAFINYLIEWVKQAPGQGLEWIGVINPGSGGTNYNEKFKGKATLTADKSSSTAYMELSSLRSDDSAVYFCARGDDYDDYTMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, synthetic construct
EIVLTQSPATLSLSPGERATLSCRASQSISNNLHWYQQKPGQAPRLLIKYASQSESGIPSRFSGSGSGTDFTLTISSLEPEDFGMYFCQQSNSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 447 aa, synthetic construct
QVQLVQSGAEVKKPGSSVKVSCKASGYAFINYLIEWVKQAPGQGLEWIGVINPGSGGTNYNEKFKGRATLTADKSTSTAYMELSSLRSEDTAVYFCARGDDYDDYTMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 214 aa, synthetic construct
DIVLTQSPATLSLSPGERATLSCRASQSISNNLHWYQQKPGQAPRLLIKYASQSMSGIPSRFSGSGSGTDFTLTISSLEPEDFGMYFCQQSNSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 447 aa, synthetic construct; identical to SEQ ID NO: 13

SEQ ID NO: 16 - protein, 214 aa, synthetic construct
EIVLTQSPATLSLSPGERATLSCRASQSISNNLHWYQQKPGQAPRLLIKYASQSMSGIPSRFSGSGSGTDFTLTISSLEPEDFGMYFCQQSNSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 447 aa, synthetic construct; identical to SEQ ID NO: 13

SEQ ID NO: 18 - protein, 214 aa, synthetic construct; identical to SEQ ID NO: 12

SEQ ID NO: 19 - protein, 447 aa, synthetic construct
QVQLVQSGAEVKKPGSSVKVSCKASGYAFINYLIEWVKQAPGQGLEWIGVINPGSGGTNYNEKFKGRVTITADKSTSTAYMELSSLRSEDTAVYYCARGDDYDDYTMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, synthetic construct; identical to SEQ ID NO: 12

SEQ ID NO: 21 - protein, 120 aa, synthetic construct
QVQLQQSGAELVRPGTSVKVSCKASGYAFINYLIEWVKQRPGQGLEWIGVINPGSGGTNYNEKFKGKATLTADKSSSTAYMQLSSLTSDDSAVYFCARGDDYDDYTMDYWGQGTSVTVSS

SEQ ID NO: 22 - protein, 120 aa, synthetic construct
QVQLQQSGAELKRPGSSVKVSCKASGYAFINYLIEWVKQAPGQGLEWIGVINPGSGGTNYNEKFKGKATLTADKSSSTAYMELSSLRSDDSAVYFCARGDDYDDYTMDYWGQGTSVTVSS

SEQ ID NO: 23 - protein, 120 aa, synthetic construct
QVQLVQSGAEVKKPGSSVKVSCKASGYAFINYLIEWVKQAPGQGLEWIGVINPGSGGTNYNEKFKGRATLTADKSTSTAYMELSSLRSEDTAVYFCARGDDYDDYTMDYWGQGTLVTVSS

SEQ ID NO: 24 - protein, 120 aa, synthetic construct
QVQLVQSGAEVKKPGSSVKVSCKASGYAFINYLIEWVKQAPGQGLEWIGVINPGSGGTNYNEKFKGRVTITADKSTSTAYMELSSLRSEDTAVYYCARGDDYDDYTMDYWGQGTLVTVSS

SEQ ID NO: 25 - protein, 107 aa, synthetic construct
DIVLSQSPATLSVTPGDSVSLSCRASQSISNNLHWYQQKSHESPRLLIKYASQSMSGIPSRFSGSGSGTDFTLSINSVETEDFGMYFCQQSNSWPLTFGAGTKLELK

SEQ ID NO: 26 - protein, 107 aa, synthetic construct
DIVLTQSPATLSLSPGERATLSCRASQSISNNLHWYQQKPGQAPRLLIKYASQSMSGIPSRFSGSGSGTDFTLTISSLEPEDFGMYFCQQSNSWPLTFGQGTKLEIK

SEQ ID NO: 27 - protein, 107 aa, synthetic construct
EIVLTQSPATLSLSPGERATLSCRASQSISNNLHWYQQKPGQAPRLLIKYASQSMSGIPSRFSGSGSGTDFTLTISSLEPEDFGMYFCQQSNSWPLTFGQGTKLEIK

SEQ ID NO: 28 - protein, 107 aa, synthetic construct
EIVLTQSPATLSLSPGERATLSCRASQSISNNLHWYQQKPGQAPRLLIKYASQSESGIPSRFSGSGSGTDFTLTISSLEPEDFGMYFCQQSNSWPLTFGQGTKLEIK

(19) United States
(12) Patent Application Publication
(10) Pub. No.: US 2026/0234267 A1
(43) Pub. Date: Aug. 13, 2026

(54) HUMANIZED ANTI-CD11B ANTIBODIES AND METHODS OF USE THEREOF

(71) Applicant: KAIGENE, INC., Rockville, MD (US)

(72) Inventors: Minjae SHIN, Rockville, MD (US); Kyunghyun MIN, Rockville, MD (US); JongJoo LEE, Rockville, MD (US); Munkyung KIM, Rockville, MD (US); Suyeun HAN, Rockville, MD (US); Junho CHO, Rockville, MD (US)

(73) Assignee: KAIGENE, INC., Rockville, MD (US)

(21) Appl. No.: 19/160,406

(22) PCT Filed: Feb. 27, 2024
(86) PCT No.: PCT/US2024/017561

§ 371 Date: Aug. 27, 2025

Related U.S. Application Data
(60) Provisional application No. 63/487,174, filed on Feb. 27, 2023.

(51) Int. Cl. C07K 16/28

(52) U.S. Cl. CPC C07K 16/2845; C07K 2317/24

(57) ABSTRACT

The present disclosure provides humanized anti-CD11b antibodies and methods of use thereof.

### CROSS-REFERENCE TO RELATED APPLICATION

This application claims the benefit of U.S. Provisional Application No. 63/487,174, which was filed on Feb. 27, 2024, and is expressly incorporated by reference herein in its entirety.

### REFERENCE TO AN ELECTRONIC SEQUENCE LISTING

The contents of the electronic sequence listing (044546.00003SequenceListing.xml, Size: 32 kB; and Date of Creation: Feb. 27, 2024) is herein incorporated by reference in its entirety.

### TECHNICAL FIELD

The present disclosure relates to the field of immunology. In particular, the present disclosure relates to humanized anti-CD11b antibodies and methods of use thereof.

### BACKGROUND

The human gene ITGAM encodes the CD11b chain of CD11b/CD18 integrin (also called Mac-1, M02, CR3, or SLEB6; UniProt Accession P11215). Mac-1 is one of the P2 integrin family members, which consist of a common P2 subunit (e.g., CD18) non-covalently attached to an a chain (e.g., α_{L}, α_{M}, α_{X}, or α_{D}). 32 integrins are expressed on the cell surface as a transmembrane heterodimer with a large extracellular region for ligand binding, a transmembrane domain, and a short cytoplasmic tail. Mac-1 is expressed on the cell surface of monocytes, macrophages, dendritic cells, neutrophils as well as lymphocytes such as NK cells and activated T cells. More than 100 proteins have been reported as ligands of Mac-1, including ICAM-1, ICAM-2, fibrinogen, iC3b, collagen, factor X and NIF. Most ligands bind to the globular head of Mac-1, the αMI domain, while some ligands bind to other parts of Mac-1, such as β-propeller domain. Like other integrins, Mac-1 remains in an inactive, bent-closed state in the absence of stimulation. In the presence of stimuli such as inflammatory cytokines, chemokines or TLR agonists, the extracellular domains of Mac-1 are extended, allowing the headpiece access to its ligands. Recognition of ligands by Mac-1 regulates various cellular functions associated with leukocyte recruitment and host defense. See Vandendriessche et al., Front Cell Dev Biol., February 11; 9:624025 (2021); Rica et al., Front Immunol., April 29; 12:662164 (2021).

Mac-1 is the major phagocytic receptor for opsonized particles such as iC3b-coated bacteria. Mac-1 clears microorganisms and immune complexes by phagocytosis, making it an important regulator of host defense and tissue homeostasis. See Rica et al. (2021). Mac-1 is also highly expressed in microglia where Mac-1 mediated phagocytosis of weakly signaling synapses is important for neural development. See Jensen et al., “Complement receptor 3 forms a compact high-affinity complex with iC3b.” J. Immunol., June 15; 206(12):3032-3042 (2021). At the same time, iC3b/C3dg-induced binding to Mac-1 on macrophages and consequent phagocytosis also play a role in pathologies of blood and CNS where aberrant complement activation is observed. In autoimmune hemolytic anemia (“AIHA”), such as warm autoimmune hemolytic anemia (“wAIHA”) and cold agglutinin disease (“CAD”), autoantibodies specific for erythrocyte membrane proteins bind to erythrocytes, resulting in clearance of erythrocytes by macrophages in the liver or spleen (i.e., extravascular hemolysis). The main cause of paroxysmal nocturnal hemoglobinuria (“PNH”), another rare hematologic disease, is the absence of complement regulators resulting in the formation of membrane attack complexes (“MACs”) on erythrocytes and consequent intravascular hemolysis, although extravascular hemolysis through macrophage phagocytosis is also suggested to play a role in the disease progression. See Zaninoni et al., “The immunomodulatory effect and clinical efficacy of daratumumab in a patient with cold agglutinin disease.” Front Immunol., June 3; 11:946 (2020); Lin et al., “Complement C3dg-mediated erythrophagocytosis: implications for paroxysmal nocturnal hemoglobinuria.” Blood, J. American Society of Hematology 126.7: 891-894 (2015). Immune thrombocytopenia (“ITP”) is also an autoimmune hematologic disease characterized by thrombocytopenia caused by platelets targeting autoantibodies. The autoantibody-coated platelets are destroyed by macrophages in the spleen and liver via F_{c}γ and complement receptors. Moreover, recent studies have demonstrated a role of microglia Mac-1 mediated phagocytosis of neurons in neurodegenerative diseases such as Alzheimer's disease, dementia, and spinal muscular atrophy. See Jensen et al. (2021).

In addition to phagocytosis, Mac-1 also plays an important role in cell-to-cell interaction and cytokine secretion that is essential for immune cell recruitment to infected or damaged tissue and inflammatory responses. This process provides a crucial contribution to the immune defense system by eliminating pathogens and cellular debris. See Vandendriessche et al. (2021), Rica et al. (2021). However, Mac-1 mediated adhesion has been proposed as a mechanism of attachment of erythrocytes to endothelial cells in sickle cell disease. See Lombardi et al., “Factor H interferes with the adhesion of sickle red cells to vascular endothelium: a novel disease-modulating molecule,” Haematologica 104.5 (2019):919. Additionally, fibrinogen binding to CR3 on leukocytes is suggested to induce nephropathy in sickle cell disease by increasing the secretion of inflammatory cytokine and reactive oxygen species (“ROS”). See Nasimuzzaman et al., “Elimination of the fibrinogen integrin αMβ2-binding motif improves renal pathology in mice with sickle cell anemia,” Blood Advances, 3.9: 1519-1532 (2019).

### BRIEF DESCRIPTION OF EXEMPLARY ASPECTS

The association of Mac-1 with immunoinflammatory dysregulation in numerous diseases has generated interest in therapeutic agents that disrupt the interaction of Mac-1 and its ligands. Several Mac-1 antagonists have been developed in the form of small molecules, peptides, or monoclonal antibodies (“mAbs”). See Vandendriessche et al. (2021), Rica et al. (2021). Although not tested in clinical trials, Mac-1 blocking mAbs, more specifically, CD11b-blocking mAbs, such as M1/70 (Springer et al., “Monoclonal xenogeneic antibodies to murine cell surface antigens: identification of novel leukocyte differentiation antigens.” European J. Immunology, 8.8: 539-551 (1978); Rotshenker, “Microglia and macrophage activation and the regulation of complement-receptor-3 (CR3/MAC-1)-mediated myelin phagocytosis in injury and disease.” J. Molecular Neuroscience 21: 65-72 (2003)); OKM10 (Talle et al., “Patterns of antigenic expression on human monocytes as defined by monoclonal antibodies.” Cellular immunology, 78.1: 83-99 (1983); Wright et al., “Identification of the C3bi receptor of human monocytes and macrophages by using monoclonal antibodies.” PNAS 80.18: 5699-5703 (1983)); anti-Mol (Arnaout et al., “Inhibition of phagocytosis of complement C3- or immunoglobulin G-coated particles and of C3bi binding by monoclonal antibodies to a monocyte-granulocyte membrane glycoprotein (Mol).” J. Clinical Investigation, 72.1: 171-179 (1983)); MN-41 (Eddy et al., “The distribution of the CR3 receptor on human cells and tissue as revealed by a monoclonal antibody.” Clinical Immunology and Immunopathology 31.3: 371-389 (1984)); 5C6 (Rotshenker (2003)); (Rosen et al., “Monoclonal antibody to the murine type 3 complement receptor inhibits adhesion of myelomonocytic cells in vitro and inflammatory cell recruitment in vivo.” J. Experimental Medicine, 166.6: 1685-1701 (1987)); and CBRM 1/5 (Lin et al. (2015); Diamond et al., “A subpopulation of Mac-1 (CD11b/CD18) molecules mediates neutrophil adhesion to ICAM-1 and fibrinogen.” J. Cell Biology, 120.2: 545-556 (1993)) have demonstrated Mac-1 blocking effects in various in vitro settings. M1/70 and 5C6 inhibited mouse myelin phagocytosis by mouse microglia in vitro. See Rotshenker (2003). CBRM 1/5 has been shown to bind only to an activation-dependent neoepitope of CR3 and block the interaction of Mac-1 with its ligands, such as ICAM-1 and fibrinogen. See Diamond et al. (1993). Thus, CBRM 1/5 recognizes activated but not resting neutrophils and monocytes, allowing specific blockade of Mac-1 in activated cells. In vitro, CBRM 1/5 blocked phagocytosis of C3dg-coated PNH erythrocytes by monocytes. See Lin et al. (2015). However, while such results are promising, there exists a need in the art for humanized Mac-1 antibodies that may be used, e.g., as a therapeutic, without provoking an undesirable immune response and corresponding negative side effects that would otherwise detract from clinical utility.

Accordingly, the present disclosure provides modified Mac-1 mAbs (targeting the CD11b polypeptide chain of Mac-1) that may be administered as human therapeutics. Modifications include humanization, affinity maturation and Fc mutations to reduce immunogenicity, increase binding affinity and modulate Fc receptor-mediated effector functions. The present disclosure thus addresses the need for humanized antibodies capable of specifically binding to a polypeptide encoded by the human gene ITGAM (e.g., the CD11b polypeptide, which forms part of the Mac-1 heterodimer as explained above).

In a first general aspect, the disclosure provides a binding agent comprising a humanized antibody or antigen-binding fragment thereof that specifically binds to a polypeptide expressed by the human gene ITGAM.

In some aspects, the polypeptide comprises a polypeptide represented by SEQ ID NO: 1, SEQ ID NO: 2, or a fragment thereof. The fragment may comprise any portion of SEQ ID NO: 1 or SEQ ID NO: 2 (e.g., any 5, 10, 15, 20, 25, 30, 35, 40, 45, 50, 55, 60, 65, 70, 75, 80, 85, 90, 95, or 100 contiguous amino acids of either sequence, or a range with endpoints defined by any pair of the foregoing size values).

In some aspects, the binding agent comprises a full-length antibody.

In some aspects, the binding agent comprises a human IgG antibody.

In some aspects, the binding agent comprises an antibody having an Fc domain that has reduced binding to FcγR than an antibody with a wild-type Fc domain.

In some aspects, the binding agent comprises an antibody having an antibody Fc domain that has increased effector function compared to an antibody with a wild-type Fc domain.

In some aspects, the binding agent comprises a humanized antibody or antigen-binding fragment thereof bound to a detectable label.

In some aspects, the binding agent comprises a humanized antibody or antigen-binding fragment thereof bound to a conjugate comprising a cytotoxic agent.

In some aspects, the antibody or fragment thereof comprises a) a heavy chain variable domain comprising a CDR1 region having a polypeptide sequence represented by SEQ ID NO: 3, a CDR2 region having a polypeptide sequence represented by SEQ ID NO: 4, and/or a CDR3 region having a polypeptide sequence represented by SEQ ID NO: 5; and/or b) a light chain variable domain comprising a CDR1 region having a polypeptide sequence represented by SEQ ID NO: 6, a CDR2 region having a polypeptide sequence represented by SEQ ID NO: 7, and/or a CDR3 region having a polypeptide sequence represented by SEQ ID NO:8.

In some aspects, a) the CDR1, CDR2, and/or CDR3 regions of the heavy chain variable domain are represented by polypeptide sequences having at least, at most, or exactly 1, 2, 3, 4, or 5 amino acid substitutions as compared to SEQ ID NOs: 3-5, respectively; and/or b) the CDR1, CDR2, and/or CDR3 regions of the light chain variable domain are represented by polypeptide sequences having at least, at most, or exactly 1, 2, 3, 4, or 5 amino acid substitutions as compared to SEQ ID NOs: 6-8, respectively.

In some aspects, the binding agent comprises an antigen-binding fragment, wherein the fragment comprises an Fab fragment, an Fab′ fragment, an F(ab′)₂ fragment, a single-chain antibody (“scFv”), a dimerized V region fragment (“diabody”), or a disulfide-stabilized V region fragment (“dsFv”).

In some aspects, the binding agent comprises: a) a heavy chain variable region (“VH”) selected from SEQ ID NOs: 21-24; and/or b) a light chain variable region (“VL”) selected from SEQ ID NOs: 25-28.

In some aspects, the binding agent comprises: a) one or more heavy chain variable regions (“VHs”) having at least, at most, or exactly 1, 2, 3, 4, or 5 amino acid substitutions compared to any one of SEQ ID NOs: 21-24; and/or b) one or more light chain variable regions (“VLs”) having at least, at most, or exactly 1, 2, 3, 4, or 5 amino acid substitutions compared to any one of SEQ ID NOs: 25-28.

In some aspects, the binding agent comprises: a) one or more heavy chain variable regions (“VHs”) having at least 85, 86, 87, 88, 89, 90, 91, 92, 93, 94, 95, 96, 97, 98, 99, or 100% sequence identity compared to any one of SEQ ID NOs: 21-24; and/or b) one or more light chain variable regions (“VLs”) having at least 85, 86, 87, 88, 89, 90, 91, 92, 93, 94, 95, 96, 97, 98, 99, or 100% sequence identity compared to any one of SEQ ID NOs: 25-28.

In some aspects, the binding agent comprises: a) a heavy chain variable region (“VH”) comprising the polypeptide sequence of SEQ ID NO: 22, and a light chain variable region (“VL”) comprising the polypeptide sequence of SEQ ID NO: 28; b) a VH comprising the polypeptide sequence of SEQ ID NO: 23, and a VL comprising the polypeptide sequence of SEQ ID NO: 26; c) a VH comprising the polypeptide sequence of SEQ ID NO: 23, and a VL comprising the polypeptide sequence of SEQ ID NO: 27; d) a VH comprising the polypeptide sequence of SEQ ID NO: 23, and a VL comprising the polypeptide sequence of SEQ ID NO: 28; or e) a VH comprising the polypeptide sequence of SEQ ID NO: 24, and a VL comprising the polypeptide sequence of SEQ ID NO: 28.

In some aspects, the binding agent comprises: a) a heavy chain variable region (“VH”) comprising a polypeptide sequence having at least 85, 86, 87, 88, 89, 90, 91, 92, 93, 94, 95, 96, 97, 98, 99, or 100% sequence identity compared to SEQ ID NO: 22, and a light chain variable region (“VL”) comprising a polypeptide sequence having at least 85, 86, 87, 88, 89, 90, 91, 92, 93, 94, 95, 96, 97, 98, 99, or 100% sequence identity compared to SEQ ID NO: 28; b) a VH comprising a polypeptide sequence having at least 85, 86, 87, 88, 89, 90, 91, 92, 93, 94, 95, 96, 97, 98, 99, or 100% sequence identity compared to SEQ ID NO: 23, and a VL comprising a polypeptide sequence having at least 85, 86, 87, 88, 89, 90, 91, 92, 93, 94, 95, 96, 97, 98, 99, or 100% sequence identity compared to SEQ ID NO: 26; c) a VH comprising a polypeptide sequence having at least 85, 86, 87, 88, 89, 90, 91, 92, 93, 94, 95, 96, 97, 98, 99, or 100% sequence identity compared to SEQ ID NO: 23, and a VL comprising a polypeptide sequence having at least 85, 86, 87, 88, 89, 90, 91, 92, 93, 94, 95, 96, 97, 98, 99, or 100% sequence identity compared to SEQ ID NO: 27; d) a VH comprising a polypeptide sequence having at least 85, 86, 87, 88, 89, 90, 91, 92, 93, 94, 95, 96, 97, 98, 99, or 100% sequence identity compared to SEQ ID NO: 23, and a VL comprising a polypeptide sequence having at least 85, 86, 87, 88, 89, 90, 91, 92, 93, 94, 95, 96, 97, 98, 99, or 100% sequence identity compared to SEQ ID NO: 28; or e) a VH comprising a polypeptide sequence having at least 85, 86, 87, 88, 89, 90, 91, 92, 93, 94, 95, 96, 97, 98, 99, or 100% sequence identity compared to SEQ ID NO: 24, and a VL comprising a polypeptide sequence having at least 85, 86, 87, 88, 89, 90, 91, 92, 93, 94, 95, 96, 97, 98, 99, or 100% sequence identity compared to SEQ ID NO: 28.

In some aspects, the binding agent comprises: a) a heavy chain variable region (“VH”) comprising a polypeptide sequence having at least, at most, or exactly 1, 2, 3, 4, or 5 amino acid substitutions compared to SEQ ID NO: 22, and a light chain variable region (“VL”) comprising a polypeptide sequence having at least, at most, or exactly 1, 2, 3, 4, or 5 amino acid substitutions compared to SEQ ID NO: 28; b) a VH comprising a polypeptide sequence having at least, at most, or exactly 1, 2, 3, 4, or 5 amino acid substitutions compared to SEQ ID NO: 23, and a VL comprising a polypeptide sequence having at least, at most, or exactly 1, 2, 3, 4, or 5 amino acid substitutions compared to SEQ ID NO: 26; c) a VH comprising a polypeptide sequence having at least, at most, or exactly 1, 2, 3, 4, or 5 amino acid substitutions compared to SEQ ID NO: 23, and a VL comprising a polypeptide sequence having at least, at most, or exactly 1, 2, 3, 4, or 5 amino acid substitutions compared to SEQ ID NO: 27; d) a VH comprising a polypeptide sequence having at least, at most, or exactly 1, 2, 3, 4, or 5 amino acid substitutions compared to SEQ ID NO: 23, and a VL comprising a polypeptide sequence having at least, at most, or exactly 1, 2, 3, 4, or 5 amino acid substitutions compared to SEQ ID NO: 28; or e) a VH comprising a polypeptide sequence having at least, at most, or exactly 1, 2, 3, 4, or 5 amino acid substitutions compared to SEQ ID NO: 24, and a VL comprising a polypeptide sequence having at least, at most, or exactly 1, 2, 3, 4, or 5 amino acid substitutions compared to SEQ ID NO: 28.

In a second general aspect, the disclosure provides A method of treating a pathology associated with phagocytosis in a human subject, comprising administering to the subject a composition comprising a therapeutically effective amount of the binding agents described herein.

In some aspects, treating the pathology comprises reducing, eliminating, or modulating one or more symptoms of the pathology.

In some aspects, the pathology is autoimmune hemolytic anemia (“AIHA”), immune thrombocytopenia (“ITP”), sickle cell disease, or a neurodegenerative disease (e.g., Alzheimer's disease, dementia, or spinal muscular atrophy).

In a third general aspect, the disclosure provides a composition comprising any of the binding agents described herein, and a pharmaceutically acceptable carrier.

In a fourth general aspect, the disclosure provides a nucleic acid molecule encoding any of the binding agents described herein.

In some aspects, the nucleic acid comprises genomic DNA, a recombinant vector, or an mRNA.

In a fifth general aspect, the disclosure provides a host cell comprising any of the nucleic acid molecules described herein.

In some aspects, the host cell comprises an E. coli, Sf9, COS, HEK293, or CHO cell.

In a sixth general aspect, the disclosure provides a pharmaceutical composition comprising any of the nucleic acid molecules described herein, and a pharmaceutically acceptable carrier.

To the accomplishment of the foregoing and related ends, the one or more aspects comprise the features hereinafter fully described and particularly pointed out in the claims. The following description sets forth in detail certain illustrative features of the one or more aspects. These features are indicative, however, of but a few of the various ways in which the principles of various aspects may be employed, and this description is intended to include all such aspects and their equivalents.

### DESCRIPTION OF THE FIGURES

The drawings set forth herein illustrate and describe exemplary aspects of the disclosure and are not meant to limit the scope of the invention as defined by the claims.

FIG. 1 is a multiple sequence alignment (“MSA”) showing the alignment of five exemplary heavy chain variable regions (designated “VH1” through “VH5”) used in humanized anti-CD11b antibody constructs that were prepared according to the present disclosure. The sequence of each heavy chains represents a portion of SEQ ID NO: 9, 11, 13, 15, 17, or 19.

FIG. 2 is an MSA showing the alignment of six exemplary light chain variable regions (designated “VL1” through “VL6”) used in humanized anti-CD11b antibody constructs that were prepared according to the present disclosure. The sequence of each heavy chains represents a portion of SEQ ID NO: 10, 12, 14, 16, 18, or 20.

FIG. 3 is a chart showing the results of a flow cytometry analysis of activated polymorphonuclear leukocytes (“PMNs”) stained using 30 humanized anti-CD11b constructs prepared according to the present disclosure. The percentages of CD11b+ cells in live PMN were calculated by a gating based on a control, which is the cells stained with human IgG4 isotype, and are presented as means S.E.M. from four independent experiments. The white bars are CD11b+ cells in untreated white blood cells (WBCs), while the black bars are CD11b+ cells in PMA-treated WBCs.

FIG. 4 is a chart summarizing the control and experimental groups used to generate the flow cytometry study results shown in FIG. 3.

FIG. 5 and FIG. 6 are charts summarizing the results of a study that assayed the same 30 humanized anti-CD11b constructs in order to determine their effects on complement-mediated phagocytosis.

FIG. 7 is a table summarizing the results of functionality tests conducted on the same 30 humanized anti-CD11b constructs described above in connect with FIGS. 3-6. As illustrated by this table, 5 of the 30 constructs were selected as lead candidates for further testing: VH1 with VL6; VH2 with VL2; VH2 with VL3; VH2 with VL6; and VH4 with VL6.

FIG. 8 and FIG. 9 show the results of a flow cytometric analysis of cells expressing wild-type or active Mac-1 using the lead candidates described in connection with FIG. 7, plus additional controls.

FIG. 8 shows histogram summarizing the flow cytometry analysis for each test or control group, and

FIG. 9 shows mean fluorescence intensities (“MFI”) data for each test or control group.

FIG. 10 is a graph showing the results of a study that examined the dose-dependent effect of the lead candidates on complement-mediated phagocytosis.

FIG. 11 is a chart showing the results of a study that evaluated TNF-α secretion from cells treated with the lead candidates.

### DETAILED DESCRIPTION

The detailed description set forth below is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

The present disclosure provides modified (e.g., humanized) antibodies that specifically recognize at least one epitope of a polypeptide encoded by the human gene ITGAM, located at 16p11.2, which encodes the CD11b chain of CD11b/CD18 integrin (also called Mac-1, αMβ2, CR3, or SLEB6; UniProt Accession P11215, as noted above). The CD11b-integrin polypeptide pairs with the CD18-integrin polypeptide to form a functional Mac-1-integrin. Mac-1 is predominantly expressed on myeloid cells such as macrophages, neutrophils, and dendritic cells, and it binds to a wide variety of ligands, including those of the ICAM family, the complement protein iC3b and fibrinogen. Mac-1 mediates phagocytosis of iC3b-coated particles, such as apoptotic cells. It also contributes to leucocyte trafficking to sites of inflammation, through binding ICAM-ligands on blood vessel endothelium, thereby assisting in movement of neutrophils on endothelium before extravasation takes place. Mac-1 has also been reported to have numerous other ligands and therefore is considered to be highly promiscuous.

Interestingly, Mac-1 has been shown to inhibit several immunological processes. Mac-1 restricts dendritic cell maturation and function and dendritic cell induced T cell activation. In macrophages, Mac-1 restricts TLR signaling through Src/Syk signaling, which results in degradation of TLR pathway downstream signaling components myeloid differentiation primary response 88 (MyD88) and TRIF, and Mac-1 has also been implicated in dampening macrophage responses through induction of signaling inhibitors such as suppressor of cytokine signaling 3 (SOCS3) and protein A20, as well as interleukin (IL)-10.

In view of its role in various immunological processes, Mac-1 (and its CD11b chain component) presents a potential therapeutic target. Accordingly, the present disclosure provides humanized antibodies capable of specifically binding to the CD11b chain of Mac-1 (i.e., a polypeptide expressed by the ITGAM). Humanized antibodies are advantageous because when a non-human antibody such as a mouse antibody is administered to a human, it is recognized as a foreign substance and induces the generation of antibodies against proteins of the non-human animal (e.g., anti-mouse antibodies, such as Human Anti-Mouse Antibody, “HAMA”). Such antibodies enhance clearance of animal-derived antibodies from the body and reduce the therapeutic effect provides by animal-derived antibodies.

The humanized antibodies described herein may be generated by grafting one or more CDRs of a non-human antibody (e.g., any or all of the CDRs represented by SEQ ID NOs. 3-8) onto a human framework to increase the human sequence content. See Safdari et al., “Antibody humanization methods—a review and update.” Biotechnology and Genetic Engineering Reviews 29.2 (2013): 175-186. Following humanization, immunogenicity of selected leads may be assessed by ex vivo immunogenicity assay. See Jaber and Baker, “Assessment of the immunogenicity of different interferon beta-la formulations using ex vivo T-cell assays.” J. Pharmaceutical and Biomedical Analysis 43.4 (2007): 1256-1261.

The binding agents described herein comprise humanized antibodies, or antigen-binding fragments thereof, capable of specifically binding to the CD11b chain expressed by the human gene ITGAM. As described above, such antibodies and antigen-binding fragments may be administered to a human subject as a therapeutic for various conditions, and in a humanized form, elicit a reduced immune response.

In some aspects, the antibody, or antigen-binding fragment thereof may comprise one or more specific CDR regions. For example, the antibody, or antigen-binding fragment thereof may comprise: a heavy chain variable domain comprising a CDR1 region having a polypeptide sequence represented by SEQ ID NO: 3, a CDR2 region having a polypeptide sequence represented by SEQ ID NO: 4, and/or a CDR3 region having a polypeptide sequence represented by SEQ ID NO: 5. In some aspects, the antibody, or antigen-binding fragment thereof may also (or alternatively) comprise: a light chain variable domain comprising a CDR1 region having a polypeptide sequence represented by SEQ ID NO: 6, a CDR2 region having a polypeptide sequence represented by SEQ ID NO: 7, and/or a CDR3 region having a polypeptide sequence represented by SEQ ID NO: 8. It is contemplated that in still further aspects, the antibody, or antigen-binding fragment thereof may comprise a CDR region that differs from any of the CDR region sequences described in this paragraph due to one or more (e.g., 1, 2, 3, 4, or 5) amino acid substitutions as compared to any of the foregoing CDR region sequences. Any or all of the substitutions may be a conservative replacement wherein the substituted amino acid is of the same class as the original amino acid (e.g., aliphatic, hydroxyl or sulfur/selenium-containing, cyclic, aromatic, basic, or acidic and the corresponding amides).

In still further aspects, humanized binding agents within the scope of the disclosure may comprise any of the heavy chain variable region (VH) and/or any of the light chain variable region (VL) sequences described herein, or a variant thereof. For example, a VH may be selected from any one of SEQ ID NOs: 21-24; and/or a VL may be selected from any one of SEQ ID NOs: 25-28. In some cases, a humanized binding agent may comprise a VH and/or a VL that shares at least 85, 86, 87, 88, 89, 90, 91, 92, 93, 94, 95, 96, 97, 98, 99, or 100% sequence identity compared to any one of SEQ ID NOs: 21-28. In some cases, a humanized binding agent may comprise a VH and/or a VL having a polypeptide sequence having at least, at most, or exactly 1, 2, 3, 4, or 5 amino acid substitutions compared to any one of SEQ ID NOs: 25-28.

In some aspects, a humanized binding agent may comprise a pair of variable domains comprising any VH described herein paired with any VL described herein, or a variant thereof (e.g., having a sequence identity level or a number of substitutions as described above with relation to the individual variable domains). For example, a humanized binding agent may comprise a) a VH comprising the polypeptide sequence of SEQ ID NO: 22, and a VL comprising the polypeptide sequence of SEQ ID NO: 28; b) a VH comprising the polypeptide sequence of SEQ ID NO: 23, and a VL comprising the polypeptide sequence of SEQ ID NO: 26; c) a VH comprising the polypeptide sequence of SEQ ID NO: 23, and a VL comprising the polypeptide sequence of SEQ ID NO: 27; d) a VH comprising the polypeptide sequence of SEQ ID NO: 23, and a VL comprising the polypeptide sequence of SEQ ID NO: 28; or e) a VH comprising the polypeptide sequence of SEQ ID NO: 24, and a VL comprising the polypeptide sequence of SEQ ID NO: 28. These particular pairings were constructed and tested as described in further detail below.

### EXAMPLES

Various aspects of the present disclosure will be illustrated with reference to the following non-limiting example.

### Example 1: Generation of Exemplary Humanized Anti-CD11b Binding Agents

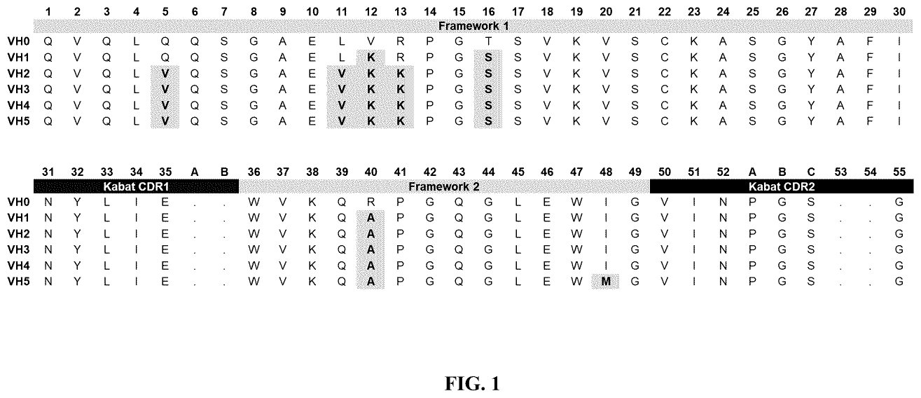

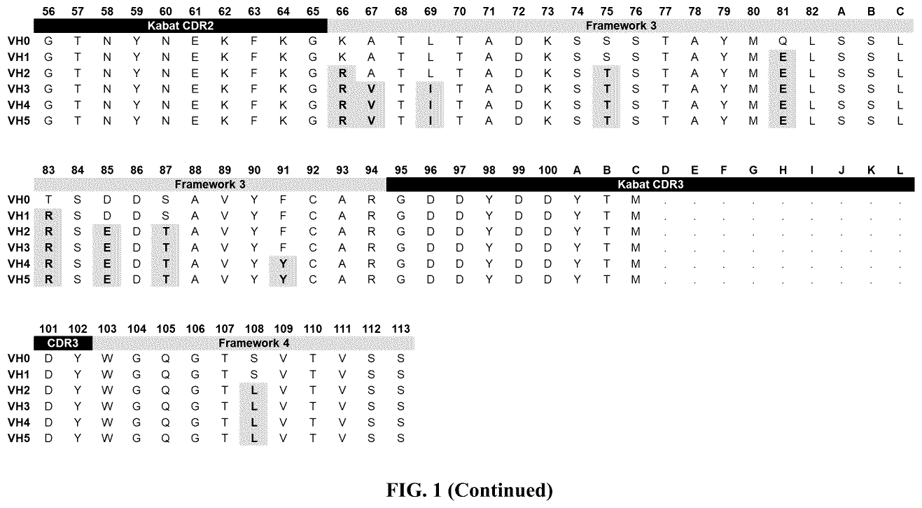

Humanization of the CBRM1/5 antibody (see Lin et al. (2015)) was conducted using Abzena's Composite Human Antibody Technology (“CHAb”). See Perry et al., “New approaches to prediction of immune responses to therapeutic proteins during preclinical development.” Drugs in R & D 9 (2008): 385-396. A total of five heavy chain (“VH1” to “VH5”) and six light chain (“VL1” to “VL6”) sequences were designed and selected for further analysis (FIG. 1). Two of the light chain designs contained substitutions to glutamic acid (E) within CDR2, at Y50 (VL5) and at M55 (VL6) to reduce a localized iTope-AI hotspot. iTope-AI scores are in silico immunogenicity scores generated from the technology for the prediction of peptide binding to a panel of MHC class II alleles (covering HLA-DR, DP and DQ).

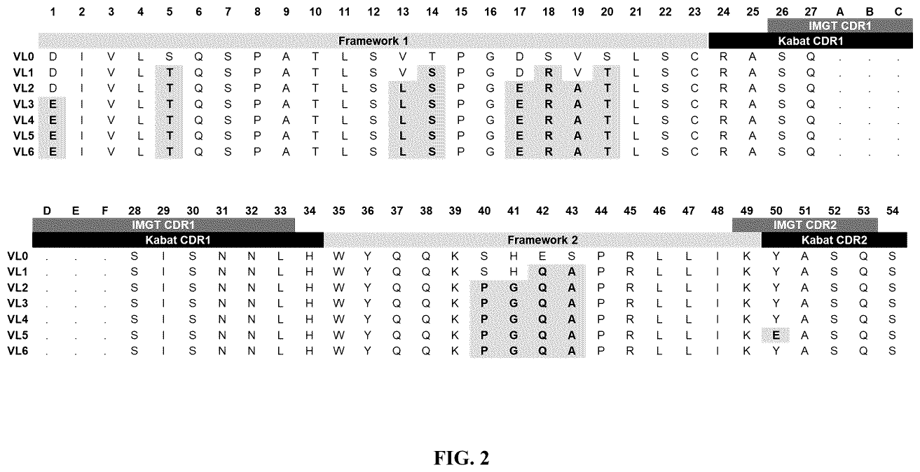

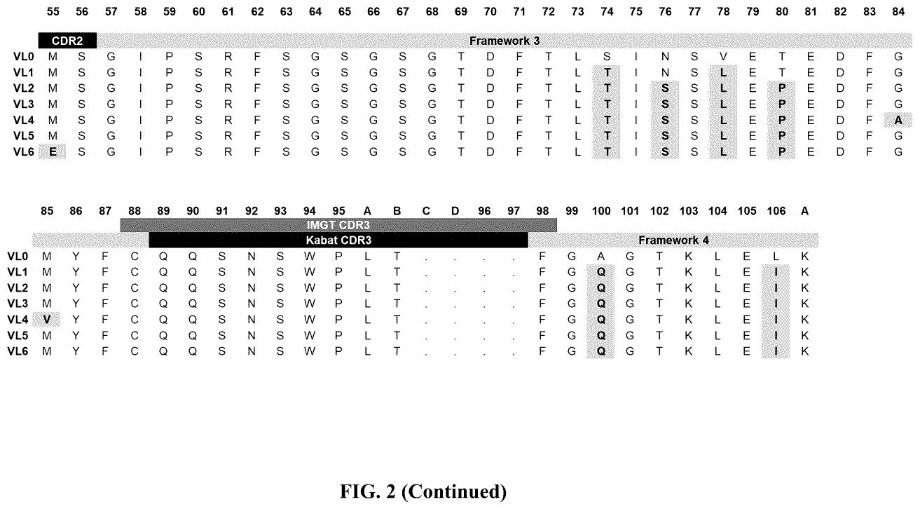

FIG. 1 and FIG. 2 show amino acid sequence alignment of CBRM1/5 and humanized antibodies. CDR definitions and protein sequence numbering according to Kabat. The variable regions of the heavy chains in humanized variants are shown in FIG. 1, and the variable regions of the light chains in humanized variants are shown in FIG. 2. “VH0” and “VL0” indicate the variable regions of heavy chain and light chain of CBRM1/5, respectively. Changes from parent (chimeric) sequence (VH0 or VL0) are highlighted in light grey. Dark gray boxes in CDR2 of the VL5 and VL6 light chains represent Y50E and M55E mutations respectively, to reduce a local iTope-AI hotspot.

TABLE 1
Summary of the homology to human germline and iTope-AI scores
for each VL domain used in candidate and/or control constructs.

| | VH0 | VHI | VH12 | VH13 | VI14 | VH5 |
|---|---|---|---|---|---|---|
| Homology to | 69.4 | 73.5 | 79.6 | 81.6 | 82.7 | 83.7 |
| human Germline | | | | | | |
| (%) | | | | | | |
| Closest Matching | IGHV1-2 | IGHV1-2 | IGHV1-69 | IGHV1-69 | IGHV1-69 | IGHV1-69 |
| Human Germline | | | | | | |
| iTope-AI Total | 107 | 62 | 46 | 37 | 36 | 34 |
| Score | | | | | | |
| Hotspot Max | 16 | 13 | 13 | 13 | 13 | 13 |

TABLE 2
Summary of the homology to human germline and iTope-AI scores
for each VL domain used in candidate and/or control constructs.

| | VL0 | VL1 | VL2 | VL3 | VL4 | VLS | VL6 |
|---|---|---|---|---|---|---|---|
| Homology to | 68.4 | 73.7 | 81.1 | 82.1 | 84.2 | 82.1 | 82.1 |
| human Germline | | | | | | | |
| (%) | | | | | | | |
| Closest Matching | IGKV6-21 | IGKV3-15 | IGKV3-11 | IGKV3-11 | IGKV3-11 | IGKV3-11 | IGKV3-11 |
| Human Germline | | | | | | | |
| iTope-AI Total | 122 | 96 | 77 | 77 | 77 | 26 | 19 |
| Score | | | | | | | |
| Hotspot Max | 28 | 28 | 28 | 28 | 28 | 6 | 5 |

Tables 1 and 2 above show the homology to human germline and iTope-AI score which represents in silico immunogenicity scores generated from CHAb technology for the prediction of peptide binding to a panel of MHC class II alleles (covering HLA-DR, DP and DQ), of each selected design. Chimeric (VH0/VL0), controls (VH0/VL1 and VH1/VL0), and 30 humanized variants generated from a combination of 5 heavy chains (VH1-VH5) and 6 light chains (VL1~VL6) were designed and their corresponding antibodies were produced in ExpiCHO cells. VH0/VL0 is a chimeric antibody that has the antigen binding fragment (Fab) of CBRM1/5 and the crystallizable fragment (Fc) of hIgG4 (S241P, L248E), and was used as a parental control.

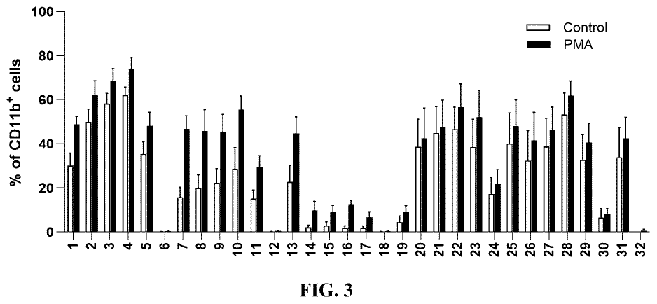

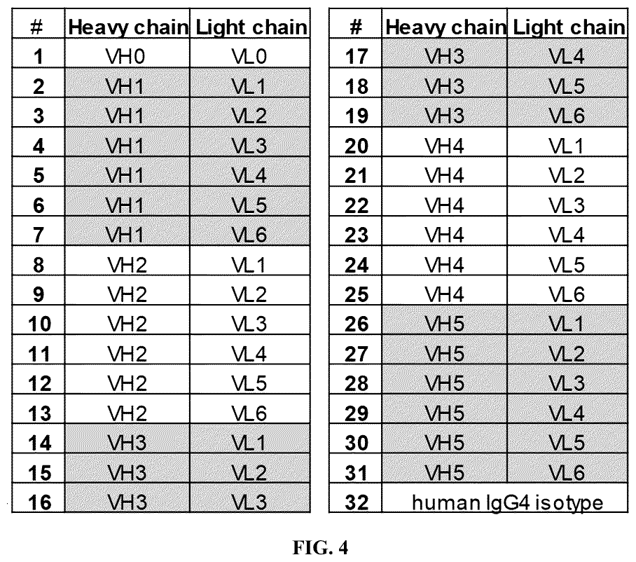

The baseline CBRM1/5 antibody can distinguish between non-activated (resting) and activated form of Mac-1. In order to validate that humanized variants retained this characteristic, WBCs were isolated from 4 healthy human donors and activated in the absence or presence of phorbol 12-myristate-13-acetate (PMA). The cells were then stained with 30 μg/mL of each of the 30 humanized variants along with control antibody (human IgG4 isotype) and analyzed using flow cytometry. The percentages of CD11b+ cells in live PMNs were calculated by a gating based on a control, which is the cells stained with human IgG4 isotype, and are presented as means S.E.M. from four independent experiments. Variants containing VL5 lost a binding to Mac-1 in polymorphonuclear leukocytes (PMNs) regardless of the pairings with any VH variant (FIGS. 3-4). Furthermore, all variants containing VH3 showed significantly reduced binding to Mac-1 (FIGS. 3-4). However, VH1/VL6 and all variants with VH2 preserved the characteristic of CBRM1/5 that can distinguish between non-activated (resting) and activated form of Mac-1, while the variants with VH4 or VH5 were likely to lose the specificity of the parental antibody, CBRM1/5 (FIGS. 3-4).

A subsequent experiment was conducted to test the functionality of 30 humanized variants by evaluating the inhibitory effect of the antibodies on phagocytosis mediated by complement. For phagocytosis, THP-1 cells differentiated with 200 ng/mL of PMA for 2 days were incubated with sheep red blood cells (RBCs) which were opsonized with 10% C5-depleted human serum. 40 μg/mL of 30 humanized variants along with chimeric controls (VH0/VL0, VH0/VL1, VH1/VL0), negative controls (mouse IgG1 isotype, human IgG4 isotype), and positive control (CBRM1/5) were treated 30 min prior to phagocytosis incubation. Statistical analysis was conducted via one-way ANOVA compared to VH0/VL0. *p≤0.05, **p≤0.01, ***p≤0.001, ****p≤0.0001

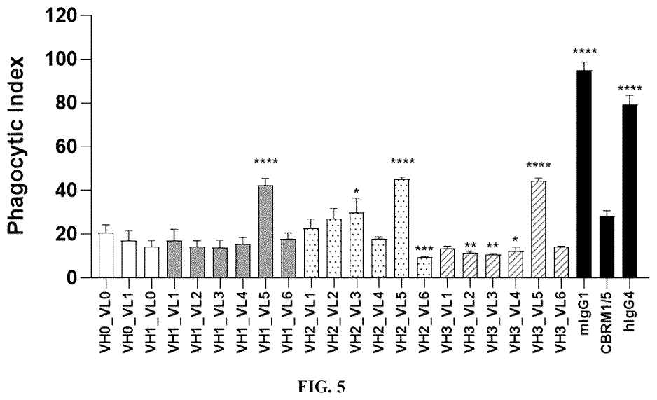

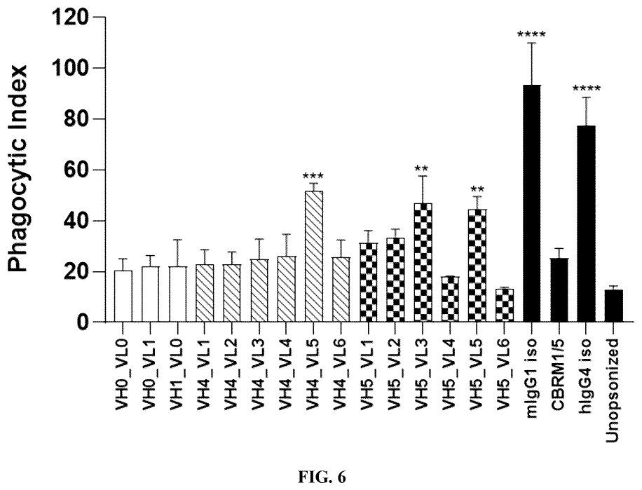

All of the humanized variants except the ones containing VL5 were found to exhibit an inhibitory effect on phagocytosis of sheep RBCs opsonized with C5-depleted human serum in differentiated THP-1 cells (FIGS. 5-6). Intriguingly, humanized variants containing VH3 exhibited reduced binding ability in PMNs (FIGS. 3-4) but inhibited complement-mediated phagocytosis as much as other variants (FIGS. 5-6).

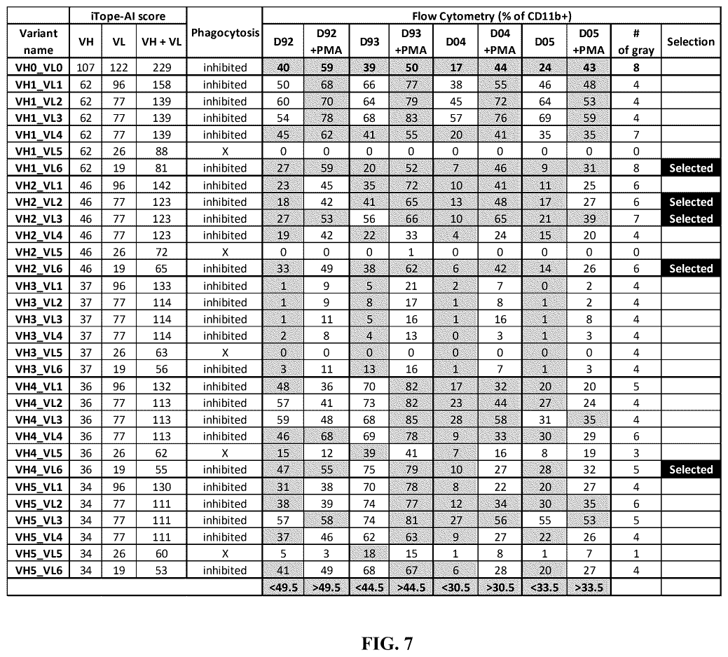

Based on the functionality results for the 30 humanized variants, which assessed their ability to bind to activated PMNs (FIGS. 3-4) and their inhibitory effects on phagocytosis (FIGS. 5-6), 5 lead humanized variants were selected: VH1/VL6, VH2/VL2, VH2/VL3, VH2/VL6, and VH4/VL6, which best retain the characteristics of original antibody (VH0/VL0). The results include iTope-AI score, phagocytic sis activity, and binding selectivity to active Mac-1 as shown in the table provided as FIG. 7. Phagocytosis data were used to determine whether each variant has an inhibitory effect on phagocytosis or not. Flow cytometry data were acquired by 4 independent experiments using untreated and PMA-treated WBCs isolated from 4 healthy human donors (D92, D93, D04, and D05). Gray shading was graded based on the criteria in the bottom row of the table. The values in the criteria were obtained as the median percentage of CD11b+ cells between untreated and PMA-treated groups stained with VH0/VL0 in each donor. The final selections indicated by black boxes in the last column of the table were made based on the iTope-AI score of VH and VL of each variant as well as its binding selectivity to active Mac-1 analyzed by flow cytometry.

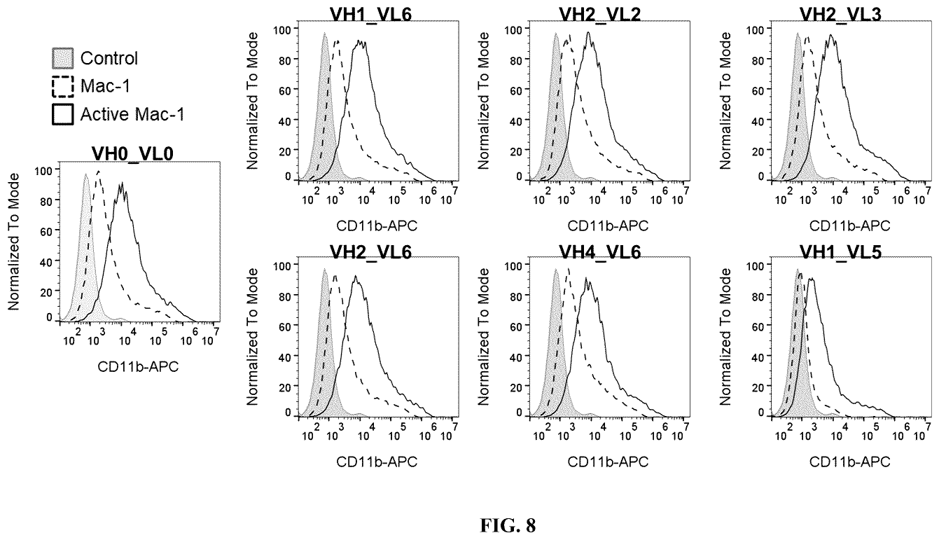

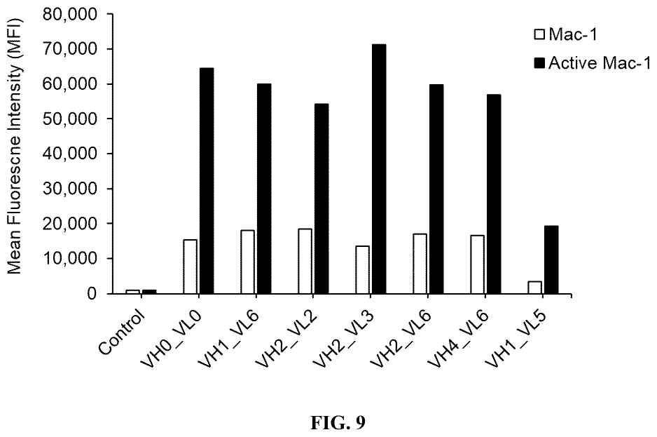

Next, the 5 lead candidate constructs (VH1/VL6, VH2/VL2, VH2/VL3, VH2/VL6, and VH4/VL6) were tested to confirm functionality. The mutation of I332G in the αMI domain of CD11b is known to induce high affinity (active) confirmation of Mac-1. See Xiong et al., “An isoleucine-based allosteric switch controls affinity and shape shifting in integrin CD11b A-domain.” J Biol Chem. 2000; 275: 38762-7. Thus, the plasmid vectors encoding CD11b WT or I332G mutant were transfected along with the plasmid vector encoding CD18 in 283FT cells to express inactive and active conformation of Mac-1. As with the VH0/VL0 variant, which is the chimeric antibody with Fab of CBRM1/5 and Fc of hIgG4 (S241P, L248E) (FIGS. 3-4), all five humanized variants bind to the active conformation of Mac-1 more efficiently than WT Mac-1, while the variant with VH1/VL5 exhibited the reduced binding to both conformations of Mac-1 (FIGS. 8-9), consistent with the results shown in FIGS. 3-4.

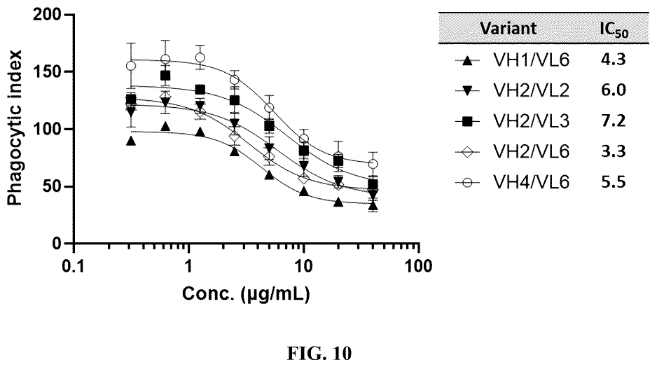

Furthermore, an experiment was conducted to evaluate the dose-dependent effect of the 5 humanized variants on phagocytosis of sheep RBCs opsonized with C5-depleted human serum in differentiated THP-1 cells. As expected, all five humanized variants exhibited a dose-dependent inhibitory effect on phagocytosis mediated by complement, although there were some differences in a half maximal inhibitory concentration (IC₅₀) among variants (FIG. 10). For phagocytosis, THP-1 cells differentiated with 200 ng/mL of PMA for 2 days were incubated with sheep RBCs which were opsonized with 10% C5-depleted human serum for 2 h. The cells were treated with 2-fold serially diluted (from 40 μg/mL to 0.313 μg/mL) antibodies of 5 humanized variants 30 min prior to phagocytosis incubation. The phagocytic index was calculated as the following equation: total number of phagocytosed RBCs/total number of macrophages*100.

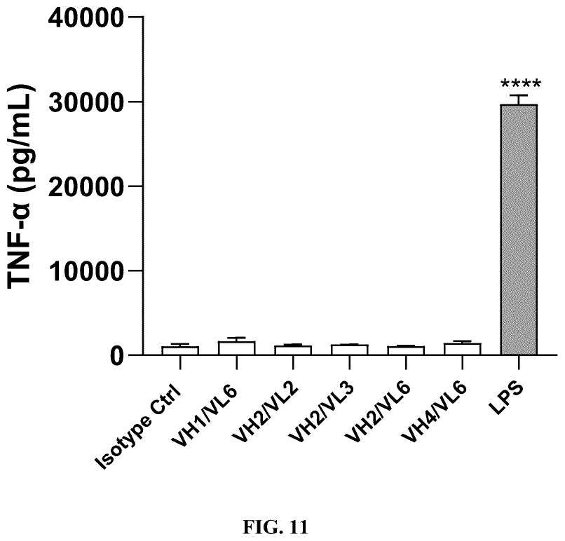

Next, an experiment was conducted to test TNF-α levels in culture supernatant from human monocyte-derived macrophages, since it has been known that phagocytic receptors clustering on macrophage membrane could lead to macrophage activation via the activation of the immunoreceptor tyrosine-based activation motif (“ITAM”) on the receptors. Human monocyte-derived macrophages were prepared by a differentiation of monocytes isolated from blood of two healthy donors with 50 ng/mL of macrophage colony-stimulating factor (“M-CSF”) for 5 days. The cells were treated with 40 μg/mL of humanized variants or isotype control or 200 ng/mL of LPS for 20 h. TNF-α levels in culture supernatant were examined by human TNF-α ELISA kit according to manufacturer's instructions. As shown by FIG. 11, in contrast to lipopolysaccharide (“LPS”), none of 5 humanized variants induced more TNF-α secretion compared to isotype control antibody, suggesting that the monoclonal antibody targeting active Mac-1 does not induce an unintended macrophage activation.

The foregoing experiments demonstrate that exemplary humanized anti-CD11b binding agents according to the disclosure are both functional and unlikely to induce an unintended macrophage activation.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc. for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

SEQUENCE LISTING

| SEQ | | |
|---|---|---|
| ID No. | Description | Sequence |
| 1 | NCBI Reference | MALRVLLLTALTLCHGFNLDTENAMTFQENARGFGQSVV |
| | Sequence: | QLQGSRVVVGAPQEIVAANQRGSLYQCDYSTGSCEPIRL |
| | NP_001139280.1 | QVPVEAVNMSLGLSLAATTSPPQLLACGPTVHQTCSENT |
| | CD11b isoform 1, | YVKGLCFLFGSNLRQQPQKFPEALRGCPQEDSDIAFLID |
| | precursor (H. | GSGSIIPHDFRRMKEFVSTVMEQLKKSKTLFSLMQYSEE |
| | sapiens) | FRIHFTFKEFQNNPNPRSLVKPITQLLGRTHTATGIRKV |
| | | VRELFNITNGARKNAFKILVVITDGEKFGDPLGYEDVIP |
| | | EADREGVIRYVIGVGDAFRSEKSRQELNTIASKPPRDHV |
| | | FQVNNFEALKTIQNQLREKIFAIEGTQTGSSSSFEHEMS |
| | | QEGFSAAITSNGPLLSTVGSYDWAGGVFLYTSKEKSTFI |
| | | NMTRVDSDMNDAYLGYAAAIILRNRVQSLVLGAPRYQHI |
| | | GLVAMFRQNTGMWESNANVKGTQIGAYFGASLCSVDVDS |
| | | NGSTDLVLIGAPHYYEQTRGGQVSVCPLPRGQRARWQCD |
| | | AVLYGEQGQPWGRFGAALTVLGDVNGDKLTDVAIGAPGE |
| | | EDNRGAVYLFHGTSGSGISPSHSQRIAGSKLSPRLQYFG |
| | | QSLSGGQDLTMDGLVDLTVGAQGHVLLLRSQPVLRVKAI |
| | | MEFNPREVARNVFECNDQVVKGKEAGEVRVCLHVQKSTR |
| | | DRLREGQIQSVVTYDLALDSGRPHSRAVFNETKNSTRRQ |
| | | TQVLGLTQTCETLKLQLPNCIEDPVSPIVLRLNFSLVGT |
| | | PLSAFGNLRPVLAEDAQRLFTALFPFEKNCGNDNICQDD |
| | | LSITFSFMSLDCLVVGGPREFNVTVTVRNDGEDSYRTQV |
| | | TFFFPLDLSYRKVSTLQNQRSQRSWRLACESASSTEVSG |
| | | ALKSTSCSINHPIFPENSEVTFNITFDVDSKASLGNKLL |
| | | LKANVTSENNMPRTNKTEFQLELPVKYAVYMVVTSHGVS |
| | | TKYLNFTASENTSRVMQHQYQVSNLGQRSLPISLVFLVP |
| | | VRLNQTVIWDRPQVTFSENLSSTCHTKERLPSHSDFLAE |
| | | LRKAPVVNCSIAVCQRIQCDIPFFGIQEEFNATLKGNLS |
| | | FDWYIKTSHNHLLIVSTAEILFNDSVFTLLPGQGAFVRS |
| | | QTETKVEPFEVPNPLPLIVGSSVGGLLLLALITAALYKL |
| | | GFFKRQYKDMMSEGGPPGAEPQ |
| 2 | NCBI Reference | MALRVLLLTALTLCHGFNLDTENAMTFQENARGFGQSVV |
| | Sequence: | QLQGSRVVVGAPQEIVAANQRGSLYQCDYSTGSCEPIRL |
| | NP_000623.2 | QVPVEAVNMSLGLSLAATTSPPQLLACGPTVHQTCSENT |
| | CD11b isoform 2, | YVKGLCFLFGSNLRQQPQKFPEALRGCPQEDSDIAFLID |
| | precursor (H. | GSGSIIPHDFRRMKEFVSTVMEQLKKSKTLFSLMQYSEE |
| | sapiens) | FRIHFTFKEFQNNPNPRSLVKPITQLLGRTHTATGIRKV |
| | | VRELFNITNGARKNAFKILVVITDGEKFGDPLGYEDVIP |
| | | EADREGVIRYVIGVGDAFRSEKSRQELNTIASKPPRDHV |
| | | FQVNNFEALKTIQNQLREKIFAIEGTQTGSSSSFEHEMS |
| | | QEGFSAAITSNGPLLSTVGSYDWAGGVFLYTSKEKSTFI |
| | | NMTRVDSDMNDAYLGYAAAIILRNRVQSLVLGAPRYQHI |
| | | GLVAMFRQNTGMWESNANVKGTQIGAYFGASLCSVDVDS |
| | | NGSTDLVLIGAPHYYEQTRGGQVSVCPLPRGRARWQCDA |
| | | VLYGEQGQPWGRFGAALTVLGDVNGDKLTDVAIGAPGEE |
| | | DNRGAVYLFHGTSGSGISPSHSQRIAGSKLSPRLQYFGQ |
| | | SLSGGQDLTMDGLVDLTVGAQGHVLLLRSQPVLRVKAIM |
| | | EFNPREVARNVFECNDQVVKGKEAGEVRVCLHVQKSTRD |
| | | RLREGQIQSVVTYDLALDSGRPHSRAVFNETKNSTRRQT |
| | | QVLGLTQTCETLKLQLPNCIEDPVSPIVLRLNFSLVGTP |
| | | LSAFGNLRPVLAEDAQRLFTALFPFEKNCGNDNICQDDL |
| | | SITFSFMSLDCLVVGGPREFNVTVTVRNDGEDSYRTQVT |
| | | FFFPLDLSYRKVSTLQNQRSQRSWRLACESASSTEVSGA |
| | | LKSTSCSINHPIFPENSEVTFNITFDVDSKASLGNKLLL |
| | | KANVTSENNMPRTNKTEFQLELPVKYAVYMVVTSHGVST |
| | | KYLNFTASENTSRVMQHQYQVSNLGQRSLPISLVFLVPV |
| | | RLNQTVIWDRPQVTFSENLSSTCHTKERLPSHSDFLAEL |
| | | RKAPVVNCSIAVCQRIQCDIPFFGIQEEFNATLKGNLSF |
| | | DWYIKTSHNHLLIVSTAEILFNDSVFTLLPGQGAFVRSQ |
| | | TETKVEPFEVPNPLPLIVGSSVGGLLLLALITAALYKLG |
| | | FFKRQYKDMMSEGGPPGAEPQ |
| 3 | Exemplary Humanized | NYLIE |
| | anti-CD11b Heavy | |
| | Chain Variable Domain | |
| | CDR1 | |
| 4 | Exemplary Humanized | VINPGSGGTNYNEKFKG |
| | anti-CD11b Heavy | |
| | Chain Variable Domain | |
| | CDR2 | |
| 5 | Exemplary Humanized | GDDYDDYTMDY |
| | anti-CD11b Heavy | |
| | Chain Variable Domain | |
| | CDR3 | |
| 6 | Exemplary Humanized | RASQSISNNLH |
| | anti-CD11b Light | |
| | Chain Variable Domain | |
| | CDR1 | |
| 7 | Exemplary Humanized | YASQSMS |
| | anti-CD11b Light | |
| | Chain Variable Domain | |
| | CDR2 | |
| 8 | Exemplary Humanized | QQSNSWPLT |
| | anti-CD11b Light | |
| | Chain Variable Domain | |
| | CDR3 | |
| 9 | Synthetic Construct | QVQLQQSGAELVRPGTSVKVSCKASGYAFINYLIEWVKQ |
| | VH0/VL0, Heavy | RPGQGLEWIGVINPGSGGTNYNEKFKGKATLTADKSSST |
| | Chain | AYMQLSSLTSDDSAVYFCARGDDYDDYTMDYWGQGTSVT |
| | Variable region (bold); | VSSASTKGPSVFPLAPCSRSTSESTAALGCLVKDYFPEP |
| | constant region of | VTVSWNSGALTSGVHTFPAVLQSSGLYSLSSVVTVPSSS |
| | IgG4, with S241P and | LGTKTYTCNVDHKPSNTKVDKRVESKYGPPCPPCPAPEF |
| | L248E mutated (plain | EGGPSVFLFPPKPKDTLMISRTPEVTCVVVDVSQEDPEV |
| | text). | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 10 | Synthetic Construct | DIVLSQSPATLSVTPGDSVSLSCRASQSISNNLHWYQQK |
| | VH0/VL0, Light Chain | SHESPRLLIKYASQSMSGIPSRFSGSGSGTDFTLSINSV |
| | Variable region (bold); | ETEDFGMYFCQQSNSWPLTFGAGTKLELKRTVAAPSVFI |
| | constant region of | FPPSDEQLKSGTASVVCLLNNFYPREAKVQWKVDNALQS |
| | IgG4, with S241P and | GNSQESVTEQDSKDSTYSLSSTLTLSKADYEKHKVYACE |
| | L248E mutated (plain | VTHQGLSSPVTKSFNRGEC |
| | text). | |
| 11 | Synthetic Construct | QVQLQQSGAELKRPGSSVKVSCKASGYAFINYLIEWVKQ |
| | VH1/VL6, Heavy | APGQGLEWIGVINPGSGGTNYNEKFKGKATLTADKSSST |
| | Chain | AYMELSSLRSDDSAVYFCARGDDYDDYTMDYWGQGTSVT |
| | Variable region (bold); | VSSASTKGPSVFPLAPCSRSTSESTAALGCLVKDYFPEP |
| | constant region of | VTVSWNSGALTSGVHTFPAVLQSSGLYSLSSVVTVPSSS |
| | IgG4, with S241P and | LGTKTYTCNVDHKPSNTKVDKRVESKYGPPCPPCPAPEF |
| | L248E mutated (plain | EGGPSVFLFPPKPKDTLMISRTPEVTCVVVDVSQEDPEV |
| | text). | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 12 | Synthetic Construct | EIVLTQSPATLSLSPGERATLSCRASQSISNNLHWYQQK |
| | VH1/VL6, Light Chain | PGQAPRLLIKYASQSESGIPSRFSGSGSGTDFTLTISSL |
| | Variable region (bold); | EPEDFGMYFCQQSNSWPLTFGQGTKLEIKRTVAAPSVFI |
| | constant region of | FPPSDEQLKSGTASVVCLLNNFYPREAKVQWKVDNALQS |
| | IgG4, with S241P and | GNSQESVTEQDSKDSTYSLSSTLTLSKADYEKHKVYACE |
| | L248E mutated (plain | VTHQGLSSPVTKSFNRGEC |
| | text). | |
| 13 | Synthetic Construct | QVQLVQSGAEVKKPGSSVKVSCKASGYAFINYLIEWVKQ |
| | VH2/VL2, Heavy | APGQGLEWIGVINPGSGGTNYNEKFKGRATLTADKSTST |
| | Chain | AYMELSSLRSEDTAVYFCARGDDYDDYTMDYWGQGTLVT |
| | Variable region (bold); | VSSASTKGPSVFPLAPCSRSTSESTAALGCLVKDYFPEP |
| | constant region of | VTVSWNSGALTSGVHTFPAVLQSSGLYSLSSVVTVPSSS |
| | IgG4, with S241P and | LGTKTYTCNVDHKPSNTKVDKRVESKYGPPCPPCPAPEF |
| | L248E mutated (plain | EGGPSVFLFPPKPKDTLMISRTPEVTCVVVDVSQEDPEV |
| | text). | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 14 | Synthetic Construct | DIVLTQSPATLSLSPGERATLSCRASQSISNNLHWYQQK |
| | VH2/VL2, Light Chain | PGQAPRLLIKYASQSMSGIPSRFSGSGSGTDFTLTISSL |
| | Variable region (bold); | EPEDFGMYFCQQSNSWPLTFGQGTKLEIKRTVAAPSVFI |
| | constant region of | FPPSDEQLKSGTASVVCLLNNFYPREAKVQWKVDNALQS |
| | IgG4, with S241P and | GNSQESVTEQDSKDSTYSLSSTLTLSKADYEKHKVYACE |
| | L248E mutated (plain | VTHQGLSSPVTKSFNRGEC |
| | text). | |
| 15 | Synthetic Construct | QVQLVQSGAEVKKPGSSVKVSCKASGYAFINYLIEWVKQ |
| | VH2/VL3, Heavy | APGQGLEWIGVINPGSGGTNYNEKFKGRATLTADKSTST |
| | Chain | AYMELSSLRSEDTAVYFCARGDDYDDYTMDYWGQGTLVT |
| | Variable region (bold); | VSSASTKGPSVFPLAPCSRSTSESTAALGCLVKDYFPEP |
| | constant region of | VTVSWNSGALTSGVHTFPAVLQSSGLYSLSSVVTVPSSS |
| | IgG4, with S241P and | LGTKTYTCNVDHKPSNTKVDKRVESKYGPPCPPCPAPEF |
| | L248E mutated (plain | EGGPSVFLFPPKPKDTLMISRTPEVTCVVVDVSQEDPEV |
| | text). | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 16 | Synthetic Construct | EIVLTQSPATLSLSPGERATLSCRASQSISNNLHWYQQK |
| | VH2/VL3, Light Chain | PGQAPRLLIKYASQSMSGIPSRFSGSGSGTDFTLTISSL |
| | Variable region (bold); | EPEDFGMYFCQQSNSWPLTFGQGTKLEIKRTVAAPSVFI |
| | constant region of | FPPSDEQLKSGTASVVCLLNNFYPREAKVQWKVDNALQS |
| | IgG4, with S241P and | GNSQESVTEQDSKDSTYSLSSTLTLSKADYEKHKVYACE |
| | L248E mutated (plain | VTHQGLSSPVTKSFNRGEC |
| | text). | |
| 17 | Synthetic Construct | QVQLVQSGAEVKKPGSSVKVSCKASGYAFINYLIEWVKQ |
| | VH2/VL6, Heavy | APGQGLEWIGVINPGSGGTNYNEKFKGRATLTADKSTST |
| | Chain | AYMELSSLRSEDTAVYFCARGDDYDDYTMDYWGQGTLVT |
| | Variable region (bold); | VSSASTKGPSVFPLAPCSRSTSESTAALGCLVKDYFPEP |
| | constant region of | VTVSWNSGALTSGVHTFPAVLQSSGLYSLSSVVTVPSSS |
| | IgG4, with S241P and | LGTKTYTCNVDHKPSNTKVDKRVESKYGPPCPPCPAPEF |
| | L248E mutated (plain | EGGPSVFLFPPKPKDTLMISRTPEVTCVVVDVSQEDPEV |
| | text). | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 18 | Synthetic Construct | EIVLTQSPATLSLSPGERATLSCRASQSISNNLHWYQQK |
| | VH2/VL6, Light Chain | PGQAPRLLIKYASQSESGIPSRFSGSGSGTDFTLTISSL |
| | Variable region (bold); | EPEDFGMYFCQQSNSWPLTFGQGTKLEIKRTVAAPSVFI |
| | constant region of | FPPSDEQLKSGTASVVCLLNNFYPREAKVQWKVDNALQS |
| | IgG4, with S241P and | GNSQESVTEQDSKDSTYSLSSTLTLSKADYEKHKVYACE |
| | L248E mutated (plain | VTHQGLSSPVTKSFNRGEC |
| | text). | |
| 19 | Synthetic Construct | QVQLVQSGAEVKKPGSSVKVSCKASGYAFINYLIEWVKQ |
| | VH4/VL6, Heavy | APGQGLEWIGVINPGSGGTNYNEKFKGRVTITADKSTST |
| | Chain | AYMELSSLRSEDTAVYYCARGDDYDDYTMDYWGQGTLVT |
| | Variable region (bold); | VSSASTKGPSVFPLAPCSRSTSESTAALGCLVKDYFPEP |
| | constant region of | VTVSWNSGALTSGVHTFPAVLQSSGLYSLSSVVTVPSSS |
| | IgG4, with S241P and | LGTKTYTCNVDHKPSNTKVDKRVESKYGPPCPPCPAPEF |
| | L248E mutated (plain | EGGPSVFLFPPKPKDTLMISRTPEVTCVVVDVSQEDPEV |
| | text). | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 20 | Synthetic Construct | EIVLTQSPATLSLSPGERATLSCRASQSISNNLHWYQQK |
| | VH4/VL6, Light Chain | PGQAPRLLIKYASQSESGIPSRFSGSGSGTDFTLTISSL |
| | Variable region (bold); | EPEDFGMYFCQQSNSWPLTFGQGTKLEIKRTVAAPSVFI |
| | constant region of | FPPSDEQLKSGTASVVCLLNNFYPREAKVQWKVDNALQS |
| | IgG4, with S241P and | GNSQESVTEQDSKDSTYSLSSTLTLSKADYEKHKVYACE |
| | L248E mutated (plain | VTHQGLSSPVTKSFNRGEC |
| | text). | |
| 21 | Heavy Chain Variable | QVQLQQSGAELVRPGTSVKVSCKASGYAFINYLIEWVKQ |
| | Region “VH0” | RPGQGLEWIGVINPGSGGTNYNEKFKGKATLTADKSSST |
| | | AYMQLSSLTSDDSAVYFCARGDDYDDYTMDYWGQGTSVT |
| | | VSS |
| 22 | Heavy Chain Variable | QVQLQQSGAELKRPGSSVKVSCKASGYAFINYLIEWVKQ |
| | Region “VH1” | APGQGLEWIGVINPGSGGTNYNEKFKGKATLTADKSSST |
| | | AYMELSSLRSDDSAVYFCARGDDYDDYTMDYWGQGTSVT |
| | | VSS |
| 23 | Heavy Chain Variable | QVQLVQSGAEVKKPGSSVKVSCKASGYAFINYLIEWVKQ |
| | Region “VH2” | APGQGLEWIGVINPGSGGTNYNEKFKGRATLTADKSTST |
| | | AYMELSSLRSEDTAVYFCARGDDYDDYTMDYWGQGTLVT |
| | | VSS |
| 24 | Heavy Chain Variable | QVQLVQSGAEVKKPGSSVKVSCKASGYAFINYLIEWVKQ |
| | Region “VH4” | APGQGLEWIGVINPGSGGTNYNEKFKGRVTITADKSTST |
| | | AYMELSSLRSEDTAVYYCARGDDYDDYTMDYWGQGTLVT |
| | | VSS |
| 25 | Light Chain Variable | DIVLSQSPATLSVTPGDSVSLSCRASQSISNNLHWYQQK |
| | Region “VL0” | SHESPRLLIKYASQSMSGIPSRFSGSGSGTDFTLSINSV |
| | | ETEDFGMYFCQQSNSWPLTFGAGTKLELK |
| 26 | Light Chain Variable | DIVLTQSPATLSLSPGERATLSCRASQSISNNLHWYQQK |
| | Region “VL2” | PGQAPRLLIKYASQSMSGIPSRFSGSGSGTDFTLTISSL |
| | | EPEDFGMYFCQQSNSWPLTFGQGTKLEIK |
| 27 | Light Chain Variable | EIVLTQSPATLSLSPGERATLSCRASQSISNNLHWYQQK |
| | Region “VL3” | PGQAPRLLIKYASQSMSGIPSRFSGSGSGTDFTLTISSL |
| | | EPEDFGMYFCQQSNSWPLTFGQGTKLEIK |
| 28 | Light Chain Variable | EIVLTQSPATLSLSPGERATLSCRASQSISNNLHWYQQK |
| | Region “VL6” | PGQAPRLLIKYASQSESGIPSRFSGSGSGTDFTLTISSL |
| | | EPEDFGMYFCQQSNSWPLTFGQGTKLEIK |

What is claimed is:

1. A binding agent comprising a humanized antibody or antigen-binding fragment thereof that specifically binds to a polypeptide expressed by the human gene ITGAM.

2. The binding agent of claim 1, wherein the polypeptide expressed by the human gene ITGAM comprises a polypeptide represented by SEQ ID NO:1, SEQ ID NO: 2, or a fragment thereof.

3. The binding agent of claim 1, wherein the binding agent comprises a full-length antibody, a human IgG antibody, an antibody having an FE domain that has reduced binding to FcγR than an antibody with a wild-type Fc domain, an antibody having an antibody FE domain that has increased effector function compared to an antibody with a wild-type FE domain, a humanized antibody or antigen-binding fragment thereof bound to a detectable label, or a humanized antibody or antigen-binding fragment thereof bound to a conjugate comprising a cytotoxic agent.

4-8. (canceled)

9. The binding agent of claim 1, wherein the antibody or fragment thereof comprises
a) a heavy chain variable domain comprising
a CDR1 region having a polypeptide sequence represented by SEQ ID NO: 3,
a CDR2 region having a polypeptide sequence represented by SEQ ID NO: 4, and/or
a CDR3 region having a polypeptide sequence represented by SEQ ID NO: 5; and/or
b) a light chain variable domain comprising
a CDR1 region having a polypeptide sequence represented by SEQ ID NO: 6,
a CDR2 region having a polypeptide sequence represented by SEQ ID NO: 7, and/or
a CDR3 region having a polypeptide sequence represented by SEQ ID NO: 8.

10. The binding agent of claim 9, wherein
a) the CDR1, CDR2, and/or CDR3 regions of the heavy chain variable domain are represented by polypeptide sequences having at least, at most, or exactly 1, 2, 3, 4, or 5 amino acid substitutions as compared to SEQ ID NOs: 3-5, respectively; and/or
b) the CDR1, CDR2, and/or CDR3 regions of the light chain variable domain are represented by polypeptide sequences having at least, at most, or exactly 1, 2, 3, 4, or 5 amino acid substitutions as compared to SEQ ID NOs: 6-8, respectively.

11. (canceled)

12. The binding agent of claim 1, wherein the binding agent comprises an antigen-binding fragment, wherein the fragment comprises an Fab fragment, an Fab′ fragment, an F(ab′)₂ fragment, a single-chain antibody (“scFv”), a dimerized V region fragment (“diabody”), or a disulfide-stabilized V region fragment (“dsFv”).

13. The binding agent of claim 1, wherein the binding agent comprises:
a) a heavy chain variable region (“VH”) selected from SEQ ID NOs: 21-24; and/or
b) a light chain variable region (“VL”) selected from SEQ ID NOs: 25-28.

14. The binding agent of claim 1, wherein the binding agent comprises:
a) one or more heavy chain variable regions (“VHs”) having at least, at most, or exactly 1, 2, 3, 4, or 5 amino acid substitutions compared to any one of SEQ ID NOs: 21-24; and/or
b) one or more light chain variable regions (“VLs”) having at least, at most, or exactly 1, 2, 3, 4, or 5 amino acid substitutions compared to any one of SEQ ID NOs: 25-28.

15. (canceled)

16. The binding agent of claim 1, wherein the binding agent comprises:
a) a heavy chain variable region (“VH”) comprising the polypeptide sequence of SEQ ID NO: 22, and a light chain variable region (“VL”) comprising the polypeptide sequence of SEQ ID NO: 28;
b) a VH comprising the polypeptide sequence of SEQ ID NO: 23, and a VL comprising the polypeptide sequence of SEQ ID NO: 26;
c) a VH comprising the polypeptide sequence of SEQ ID NO: 23, and a VL comprising the polypeptide sequence of SEQ ID NO: 27;
d) a VH comprising the polypeptide sequence of SEQ ID NO: 23, and a VL comprising the polypeptide sequence of SEQ ID NO: 28; or
e) a VH comprising the polypeptide sequence of SEQ ID NO: 24, and a VL comprising the polypeptide sequence of SEQ ID NO: 28.

17. (canceled)

18. The binding agent of claim 1, wherein the binding agent comprises:
a) a heavy chain variable region (“VH”) comprising a polypeptide sequence having at least, at most, or exactly 1, 2, 3, 4, or 5 amino acid substitutions compared to SEQ ID NO: 22, and a light chain variable region (“VL”) comprising a polypeptide sequence having at least, at most, or exactly 1, 2, 3, 4, or 5 amino acid substitutions compared to SEQ ID NO: 28;
b) a VH comprising a polypeptide sequence having at least, at most, or exactly 1, 2, 3, 4, or 5 amino acid substitutions compared to SEQ ID NO: 23, and a VL comprising a polypeptide sequence having at least, at most, or exactly 1, 2, 3, 4, or 5 amino acid substitutions compared to SEQ ID NO: 26;
c) a VH comprising a polypeptide sequence having at least, at most, or exactly 1, 2, 3, 4, or 5 amino acid substitutions compared to SEQ ID NO: 23, and a VL comprising a polypeptide sequence having at least, at most, or exactly 1, 2, 3, 4, or 5 amino acid substitutions compared to SEQ ID NO: 27;
d) a VH comprising a polypeptide sequence having at least, at most, or exactly 1, 2, 3, 4, or 5 amino acid substitutions compared to SEQ ID NO: 23, and a VL comprising a polypeptide sequence having at least, at most, or exactly 1, 2, 3, 4, or 5 amino acid substitutions compared to SEQ ID NO: 28; or
e) a VH comprising a polypeptide sequence having at least, at most, or exactly 1, 2, 3, 4, or 5 amino acid substitutions compared to SEQ ID NO: 24, and a VL comprising a polypeptide sequence having at least, at most, or exactly 1, 2, 3, 4, or 5 amino acid substitutions compared to SEQ ID NO: 28.

19. A method of treating a pathology associated with phagocytosis in a human subject, comprising administering to the subject a composition comprising a therapeutically effective amount of the binding agent of claim 1.

20. The method of claim 19, wherein treating the pathology comprises reducing, eliminating, or modulating one or more symptoms of the pathology.

21. The method of claim 19, wherein the pathology is autoimmune hemolytic anemia (“AIHA”), immune thrombocytopenia (ITP), sickle cell disease, a neurodegenerative disease, a demyelinating disease.

22-24. (canceled)

25. The method of claim 2421, wherein the neurodegenerative disease is Alzheimer's disease, dementia, or spinal muscular atrophy.

26. (canceled)

27. The method of claim 2421, wherein the demyelinating disease is Chronic Inflammatory Demyelinating Polyneuropathy (“CIDP”) or Multifocal Motor Neuropathy (“MMN”).

28. A composition comprising the binding agent of claim 1 and a pharmaceutically acceptable carrier.

29. A nucleic acid molecule encoding the binding agent of claim 1.

30. (canceled)

31. A host cell comprising the nucleic acid molecule of claim 29.

32. (canceled)

33. A pharmaceutical composition comprising:
the nucleic acid molecule of claim 29, and
at least one pharmaceutically acceptable excipient.

34. (canceled)
